(19) United States
(12) Patent Application Publication
(10) Pub. No.: US 2004/0005321 A1
(43) Pub. Date: Jan. 8, 2004

(54) Use of anti-ceacam antibodies for stimulating b cells in the production of monoclonal antibodies or in immunotheraphy

(76) Inventors: Bernhard B Singer, Berlin (DE); Gediminas Greicius, Stockholm (SE)

Correspondence Address: BROWDY AND NEIMARK, P.L.L.C., 624 NINTH STREET, NW, SUITE 300, WASHINGTON, DC, 20001-5303, US

(21) Appl. No.: 10/344,036

(22) PCT Filed: Aug. 7, 2001
(86) PCT No.: PCT/SE01/01714

(30) Foreign Application Priority Data
Aug. 7, 2000 (SE) 0002835-7

(51) Int. Cl. A61K 39/395; C12P 21/04

(52) U.S. Cl. 424/146.1; 435/70.21

(57) ABSTRACT

The present invention relates to a method and kit for the production of monoclonal anitbodies using B cell stimulatory agent(s), such as antiCEACAM antibody and LPS. Furthermore, the invention relates to use of CEACAM for B cell stimulation in for example immunotherapy.

### FIELD OF THE INVENTION

The present invention relates to a method and kit for the production of monoclonal antibodies. Furthermore, the invention relates to use of the cell surface molecule CEACAM for B cell stimulation.

### BACKGROUND OF THE INVENTION

Lymphocytes play a central role in the initiation and regulation of antigen specific immune response. Hereby, the membrane-bound form of the immunoglobulin (Ig) organized in the T-cell receptor (TCR) and B-cell receptor (BCR) defines the specificity of lymphocytes. Each mature T cell and B cell carries a single specificity. The binding of an appropriate antigen to e.g. the BCR initiates clonal expansion and differentiation of B-cells into antibody secreting plasma cells. This fact led to the discovery of monoclonal antibodies (mAbs) by Köhler and Milstein in 1975 who developed a technique to generate antibody secreting hybridoma cells by fusing myeloma cells with specific immunized B-lymphocytes. In non-immune animals B lymphocytes express a broad range of B cell receptor (BCR) specificity. Therefore it is crucial to immunize animals in order to enrich for antigen specific B cell clones. The Immunization should also be repeated in order to generate antibodies of immunoglobulin class other than IgM. But even then it requires large effort to characterize for the specificity and to eliminate hybridoma cells produced as a result of the fusion of myeloma cells and non-B cells. Nowadays mAbs are the most important tools used in biomedical research, diagnosis and treatment of diseases such as infections and cancer (Green, 1999).

CEACAM1 (also known as C-CAM, BGP and CD66a) is within the CEA-subgroup a member of the immunoglobulin superfamily. CEACAM1 is abundantly expressed in epithelia and vessel endothelia, granulocytes and lymphocytes. So far the only known physiological ligand is CEACAM1 itself mediating a homophilic cell-cell adhesion. Previously it has been shown that CEACAM1 is a potent, signal-transducing molecule. The function of CEACAM1 seems to be diverse and cell type specific. In epithelial cells CEACAM1 is involved in growth control (Singer et al., 2000), while in granulocytes it mediates specific activation reactions lie the induction of the respiratory burst and the up-regulation of the integrin mediated adhesion (Skubitz et al., 2000). In T cells a TCR dependent costimulatory function could be assign to CEACAM1 (Kammerer et al. 1998). However, the common nominator for the CEACAM1 function seems to be the signaling of the cell-cell contact followed by diverse, cell type specific functional reactions.

### SUMMARY OF THE INVENTION

The present invention provides a simple and rapid way of selection of antigen specific B cell clones before the fusion. This method is based on our discovery, that the co-engagement of BCR and cell surface molecule CEACAM1 leads to an increased proliferation and prolongs the survival of activated cells and therefore leads to a dramatically increase of the amount of antigen specific hybridoma cells.

The present inventors have found that CEACAM1 is a potent BCR-costimulatory molecule. The present inventors have demonstrated that following the engagement of an appropriate antigen, CEACAM1 triggers B cell proliferation. Utilizing this effect we invented a novel method for the generation of mAbs with significant higher efficiency compared to the traditional technique or the in vitro immunization approach.

Thus, in a first aspect the invention relates to a method for production of monoclonal antibodies, comprising

immunization with an antigen for enrichment of antibody producing B cells; preparation of B cells; fusion of B cells with immortal cells to form antibody producing hybridomas, comprising the following additional step:

in vitro expansion of said B cells in the presence of B cell stimulating agent(s) before said fusion.

The B cell stimulating agent(s) comprises an antibody against CEACAM or CEACAM ligands or, or against a B cell stimulatory variant thereof, and an antigen against which said B cells are reactive. The antibody can be directed against CEACAM1 or CEACAM2, preferably CEACAM1. The immunization is in vivo or in vitro.

CEACAM ligands in accordance with the present invention include chemical agents that modulate the action of CEACAM, either through altering its biological activity or through modulation of expression, e.g., by affecting transcription or translation of the CEACAM encoding gene. CEACAM ligands include binding proteins derived for example from anti-CEACAM antibody and may be designed by structure-assisted computer modeling for example according to alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions, substrate binding region, and high antigenic index regions. Computer predictions can be made made using for example GCG-software derived from HGMP resource center Cambridge (Rice, 1995) Programe Manual for the EGCG package. (Cambridge, CB10 1RQ, England: Hinxton Hall).

Using known methods of protein engineering and recombinant DNA technology, variants may be generated to improve or alter the characteristics of the CEACAM polypeptides. For instance, one or more amino acids can be deleted from the N-terminus or C-terminus of the protein without substantial loss of biological function. The authors of Ron, J. Biol. Chem. 268 (1993), 2984-2988, reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein. Dobeli, J. Biotechnology 7 (1988), 199-216). Moreover, ample evidence demonstrates that variants often retain a biological activity similar to that of the naturally occurring protein. For example, Gayle and coworkers (J. Biol. Chem. 268 (1993); 22105-22111) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[most of the molecule could be altered with little effect on either [binding or biological activity]”; see Abstract. In fact, only 23 unique amino acid sequences, out of more than 3,500 nucleotide sequences examined, produced a protein that significantly differed in activity from wild-type. Furthermore, using the PESTFIND program (Rogers, Science 234 (1986), 364-368), PEST sequences (rich in proline, glutamic acid, serine, and threonine) can be identified, which are characteristically present in unstable proteins. Such sequences may be removed from the CEACAM proteins in order to increase the stability and optionally the activity of the proteins. Methods for introducing such modifications in the nucleic acid molecules according to the invention are well-known to the person skilled in the art.

Thus, the present invention includes the use of CEACAM polypeptide variants which show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie, Science 247 (1990), 1306-1310, wherein the authors indicate that there are two main strategies for studying the tolerance of an amino acid sequence to change.

Besides conservative amino acid substitution, variants of CEACAM include (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as an IgG Fc fusion region peptide, or leader or secretary sequence, or a sequence facilitating purification. Such variant polypeptides are deemed to be within the scope of those skilled in the art from the teachings herein. For example, CEACAM polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity; see, e.g. Pinckard, Clin. Exp. Immunol. 2 (1967), 331-340; Robbins, Diabetes 36 (1987), 838-845; Cleland, Crit. Rev. Therapeutic Drug Carrier Systems 10 (1993), 307-377.

An anti-CEACAM antibody to be used in accordance with the methods of the present invention can be a monoclonal antibody, a polyclonal antibody, a single chain antibody, human or humanized antibody, primatized, chimerized, xenogeneic or fragment thereof that specifically binds an CEACAM peptide or polypeptide also including bispecific antibody, synthetic antibody, antibody fragment, such as Fab, Fv or scFv fragments etc., or a chemically modified derivative of any of these. The general methodology for producing antibodies is well-known and has been described in, for example, Köhler and Milstein, Nature 256 (1975), 494 and reviewed in J. G. R. Hurrel, ed., “Monoclonal Hybridoma Antibodies: Techniques and Applications”, CRC Press Inc., Boco Raron, Fla. (1982), as well as that taught by L. T. Mimms et al., Virology 176 (1990), 604-619. Furthermore, antibodies or fragments thereof to the aforementioned peptides can be obtained by using methods which are described, e.g., in Harlow and Lane “Antibodies, A Laboratory Manual”, CSH Press, Cold Spring Harbor, 1988.

Further sources for the basic structure of CEACAM ligands can be employed and comprise, for example, mimetic analogs of the CEACAM polypeptide. Mimetic analogs of the CEACAM polypeptide or biologically active fragments thereof can be generated by, for example, substituting the amino acids that are expected to be essential for the biological activity with, e.g., stereoisomers, i.e. D-amino acids; see e.g., Tsukida, J. Med. Chem. 40 (1997), 3534-3541. Furthermore, in case fragments are used for the design of biologically active analogs pro-mimetic components can be incorporated into a peptide to reestablish at least some of the conformational properties that may have been lost upon removal of part of the original polypeptide; see, e.g., Nachman, Regul. Pept. 57 (1995), 359-370. Furthermore, the CEACAM polypeptide can be used to identify synthetic chemical peptide mimetics that bind to or can function as a ligand, substrate, binding partner or the receptor of the CEACAM polypeptide as effectively as does the natural polypeptide; see, e.g., Engleman, J. Clin. Invest. 99 (1997), 2284-2292. For example, folding simulations and computer redesign of structural motifs of the protein of the invention can be performed using appropriate computer programs (Olszewski, Proteins 25 (1996), 286-299; Hoffman, Comput. Appl. Biosci. 11 (1995), 675-679). Computer modeling of protein folding can be used for the conformational and energetic analysis of detailed peptide and protein models (Monge, J. Mol. Biol. 247 (1995), 995-1012; Renouf, Adv. Exp. Med. Biol. 376 (1995), 37-45). In particular, the appropriate programs can be used for the identification of interactive sites of the CEACAM polypeptide and its ligand or other interacting proteins by computer assistant searches for complementary peptide sequences (Fassina, Immunomethods 5 (1994), 114-120. Further appropriate computer systems for the design of protein and peptides are described in the prior art, for example in Berry, Biochem. Soc. Trans. 22 (1994), 1033-1036; Wodak, Ann. N.Y. Acad. Sci. 501 (1987), 1-13; Pabo, Biochemistry 25 (1986), 5987-5991. Methods for the generation and use of peptide mimetic combinatorial libraries are described in the prior art, for example in Ostresh, Methods in Enzymology 267 (1996), 220-234 and Dorner, Bioorg. Med. Chem. 4 (1996), 709-715. Furthermore, a three-dimensional and/or crystallographic structure of the CEACAM protein can be used for the design of mimetic inhibitors of the biological activity of the protein of the invention (Rose, Biochemistry 35 (1996), 12933-12944; Rutenber, Bioorg. Med. Chem. 4 (1996), 1545-1558).

The structure-based design and synthesis of low-molecular-weight synthetic molecules that mimic the activity of the native biological polypeptide is further described in, e.g., Dowd, Nature Biotechnol. 16 (1998), 190-195; Kieber-Emmons, Current Opinion Biotechnol. 8 (1997), 435-441; Moore, Proc. West Pharmacol. Soc. 40 (1997), 115-119; Mathews, Proc. West Pharmacol. Soc. 40 (1997), 121-125; Mukhija, European J. Biochem. 254 (1998), 433-438.

Recombinant CEACAM polypeptides and nucleic acid molecules can be produced by methods known to those skilled in molecular biology. For example, the choice of vectors which would depend on the function desired and include plasmids, cosmids, viruses, bacteriophages and other vectors used conventionally in genetic engineering. Methods which are well known to those skilled in the art can be used to construct various plasmids and vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1989), (1994).

The monoclonal antibody obtained by the method of the invention may be further characterized or processed to known methods. For example, in case of a rodent antibody said monoclonal antibody may be humanized and/or synthetically altered to obtain a single chain antibody, a bispecific antibody, synthetic antibody, antibody fragments, such as Fab, Fv or scFv fragments etc., or a chemically modified derivative of any of these. For these reasons, RNA encoding the light and heavy chains of the immunoglobulin can then be obtained from the cytoplasm of the hybridoma or directly from the antibody producing B cell. The 5′ end portion of the mRNA can be used to prepare cDNA to be inserted into an expression vector. The DNA encoding the antibody or its immunoglobulin chains can subsequently be expressed in cells, preferably mammalian cells. Depending on the host cell, renaturation techniques may be required to attain proper conformation of the antibody. If necessary, point substitutions seeking to optimize binding may be made in the DNA using conventional cassette mutagenesis or other protein engineering methodology such as is disclosed herein.

The production of chimeric antibodies is described, for example, in WO89/09622. Methods for the production of humanized antibodies are described in, e.g., EP-A1 0 239 400 and WO90/07861.

Antibodies obtained by the method of the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook, Molecular Cloning A Laboratory Manual, Cold Spring Harbor Laboratory (1989) N.Y.

Furthermore, a further domain may be added to the antibodies obtained by the method of the present invention by covalent or non-covalent bonds. The linkage can be based on genetic fusion according to the methods known in the art and described above or can be performed by, e.g., chemical cross-linking as described in, e.g., WO 94/04686. The additional domain present in the fusion antibody may preferably be linked by a flexible linker, advantageously a polypeptide linker, wherein said polypeptide linker comprises plural, hydrophilic, peptide-bonded amino acids of a length sufficient to span the distance between the C-terminal end of said further domain and the N-terminal end of the peptide, polypeptide or antibody or vice versa. The above described fusion protein may further comprise a cleavable linker or cleavage site for proteinases

It will be apparent to those skilled in the art that the antibodies obtained by the method of the present can be further coupled to other moieties as described above for, e.g., drug targeting and imaging applications. Such coupling may be conducted chemically after expression of the protein to site of attachment or the coupling product may be engineered into the protein of the invention at the DNA level. The DNAs are then expressed in a suitable host system, and the expressed proteins are collected and renatured, if necessary.

In a further aspect, the invention relates to a kit for production of monoclonal antibodies, comprising B cell stimulating agents) for in vitro expansion of B cells. Preferably, the B cell stimulating agent(s) comprises anti-CEACAM antibody, preferably anti-CEACAM1. In yet a further aspect, the invention relates to use of CEACAM, preferably CEACAM1, for B cell stimulation. The use may be in connection with mAb production as above or, for example, in connection with treating disorders using the specific clonal expanded B cell subpopulation in an immunotherapy. For example, in a B cell immunotherapy the specific B cell subclones of an AIDS or cancer patient are amplified in vitro and by re-infusion given to the patient to strengthen his immun defense system.

Thus CAECAM1 and ligands may be used as pharmaceutical compositions or vaccines for immuno-treatment of a subject. The pharmaceutical compositions or vaccines may be prepared using conventional carriers and excipients suitable for human use.

According to the invention, a patients specific antigen recognizing B cell subpopulations are amplified in vitro and the antibodies are then isolated and can be used either to perform immunotherapy or as a diagnostic tool for identifying antigens.

CEACAM is a specific antigen dependent co-stimulator of B cell proliferation and can be used for the amplification of specific antigen recognizing B cell subpopulations. According to the invention, you can use these cells for different applications: either for a fusion to get monoclonal antibodies or to re-infuse the amplified cells in a patient to perform immunotherapy.

Thus, the invention provides for the use of an effective amount of CEACAM, anti-CEACAM antibody and CEACAM ligand to induce and/or increase an immune response in vivo, for example, in the patient's peripheral blood, tissues or organs. CEACAM, anti-CEACAM antibody and CEACAM ligand may be used to increase the numbers of antibody producing B cells in vivo to boost a patient's immune response against existing antigens. Alternatively, CEACAM, anti-CEACAM antibody and CEACAM ligand may be administered prior to, concurrently with or subsequent to administration of an antigen to a patient for immunization purposes. Thus, as a vaccine adjuvant, CEACAM, anti-CEACAM antibody and CEACAM ligand can generate large quantities of B cells and/or intermediate cells in vivo to more effectively present the antigen. The overall response is a stronger and improved immune response and more effective immunization to the antigen. The vaccine of the invention may be administered with one or more of the molecules selected from the group consisting of GM-CSF, IL-4, TNFa, IL-3, c-kit ligand, flt-ligand and fusions of GM-CSF and IL3.

As used herein, “vaccine” means an organism or material that contains an antigen in an innocuous form. The vaccine is designed to trigger an immunoprotective response. The vaccine may be recombinant or non-recombinant. When inoculated into a non-immune host, the vaccine will provoke active immunity to the organism or material, but will not cause disease. Vaccines may take the form, for example, of a toxoid, which is defined as a toxin that has been detoxified but that still retains its major immunogenic determinants; or a killed organism, such as typhoid, cholera and poliomyelitis; or attenuated organism, that are the live, but non-virulent, forms of pathogens, or it may be antigen encoded by such organism, or it may be a live tumor cell or an antigen present on a tumor cell.

For in vivo administration to humans, CEACAM, anti-CEACAM antibody and CEACAM ligand can be formulated according to known methods used to prepare pharmaceutically useful compositions. CEACAM, anti-CEACAM antibody and CEACAM ligand be combined in admixture, either as the sole active material or with other known active materials, with pharmaceutically suitable diluents (e.g., Tris-HCl, acetate, phosphate), preservatives (e.g., Thimerosal, benzyl alcohol, parabens), emulsifiers, solubilizers, adjuvants and/or carriers. Suitable carriers and their formulations are described in Remington's Pharmaceutical Sciences, 16th ed. 1980, Mack Publishing Co. In addition, such compositions can contain CEACAM, anti-CEACAM antibody and CEACAM ligand complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance of CEACAM, anti-CEACAM antibody and CEACAM ligand.

CEACAM, anti-CEACAM antibody and CEACAM ligand can be administered topically, parenterally, or by inhalation. The term “parenteral” includes subcutaneous injections, intravenous, intramuscular, intracisternal injection, or infusion techniques. These compositions will typically contain an effective amount of the CEACAM, anti-CEACAM antibody and CEACAM ligand, alone or in combination with an effective amount of any other active material. Such dosages and desired drug concentrations contained in the compositions may vary depending upon many factors, including the intended use, patient's body weight and age, and route of administration. Preliminary doses can be determined according to animal tests, and the scaling of dosages for human administration can be performed according to art-accepted practices. Keeping the above description in mind, typical dosages of CEACAM, anti-CEACAM antibody and CEACAM ligand may range from about 10 Rg per square meter to about 1000 lig per square meter. A preferred dose range is on the order of about 100 llg per square meter to about 3(X) llg per square meter.

These and other embodiments are disclosed and encompassed by the description and Examples of the present invention. Further literature concerning any one of the antibodies, methods, uses and compounds to be employed in accordance with the present invention may be retrieved from public libraries and databases, using for example electronic devices. For example the public database “Medline” may be utilized which is available on the Internet, for example under http://www.ncbi.nlm.nih.gov/PubMed/medline.html. Further databases and addresses, such as http://www.ncbi.nlm.nih.gov/, http://www.infobiogen.fr/, http://www.fmi.ch/biology/research_tools.html, http://www.tigr.org/, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

This disclosure may best be understood in conjunction with the accompanying drawings, incorporated herein by references. Furthermore, a better understanding of the present invention and of its many advantages will be had from the following examples, given by way of illustration and are not intended as limiting.

Unless stated otherwise in the examples, all recombinant DNA techniques are performed according to protocols as described in Sambrook et al. (1989), Molecular Cloning: A Laboratory Manual. Cold Spring Harbor Laboratory Press, NY or in Volumes 1 and 2 of Ausubel et al. (1994), Current Protocols in Molecular Biology, Current Protocols.

### DETAILED DESCRIPTION OF THE INVENTION

Materials and Methods

Mice: C57BL/6 were assayed for the analyses of the CEACAM1 function. For immunizations Balb/c female mice of 6-8 weeks of age were used.

Reagents and antibodies: Lipopolysaccharide (LPS) was used from Sigma-Aldrich, IL-4 was produced by hybridoma X63 transfected with IL-4 cDNA (Karasuyama and Melchers 1988).

Anti-IgM antibodies Ak13 (Leptin et al. 1984) were prepared by ammonium sulphate precipitation from hybridoma culture supernatants and were coupled to cyanogen bromide activated Sepharose beads (Amersham Pharmacia Biotech, Uppsala, Sweden) according to manufacturers instructions. Antibodies were coupled at concentration 2 mg/ml bed vol. The Beads were stored in sterile Tris-HCl buffer (0.1 M, pH 8.0) containing 0.5 M NaCl and washed in sterile culture medium prior usage. Ak13 coupled beads were used at 0.5% bead vol/ vol. The rat anti-mouse CEACAM1 mAb (AgB10; IgG1) (Kuprina et al. 1990), a kind gift from T. D. Rudinskaya, the mAb rat anti-mouse E-cadherin (Decma1; IgG1) and the rat serum Ig were affinity purified on protein G column (Amersham Pharmacia Biotech, Uppsala, Sweden) according to the manufacturer's protocol.

Cell Proliferation assay: B-cells were resuspended to a concentration of 4×10⁵/ml, distributed at 200 μl per well in 96-well plates and stimulated as indicated. After indicated time ³H-thymidine was added to a final activity of 2 μCi/ml and the cells were cultured for a further 6 hours prior to harvesting and measurement of activity using a beta-counter (Wallac, Turku, Finland).

Cell culture conditions: B-cells were enriched from spleen cell suspensions by incubation in hybridoma supernatants containing anti-Thy-1.2 (AT83A), anti-CD4 (RL172.4) and anti-CD8 (31M) and with Low tox rabbit complement (European Saxon Ltd, Suffolk, GB) in Eagle's Balanced Salt solution (BSS, Gibco BRL, Life Technology, Paisley, UK) at 37° C. for 1 hour. Small resting B-cells were prepared by Percoll (Pharmacia, Uppsala, Sweden) gradient centrifugation. The cells from the Percoll gradient between layers 50% and 70% were washed twice in BSS and resuspended in complete RPMI 1640 medium containing 10 mM HEPES, 1 mM sodium pyruvate, 50 mM 2-mercaptoethanol, 100 IU and 100 μg/ml of penicillin-streptomycin respectively and 10% of fetal calf serum (all from GIBCO BRL, Life technology). The concentration of cells was adjusted to 1×10⁶ cell/ml, distributed into 24- or 6- well plates and cultured at 37° C. in 5% CO₂ for 4 days if not indicated otherwise.

The B cell stimulation was performed with anti-mouseCEACAM-1 mAb (AgB10) or isotype control mAb Decma1 in a concentration of 100 μg/ml, while the appropriate antigens were used at 20 μg/ml.

Immunizing mice and fusion procedure: Female Balb/C mice were injected with 20 μg antigen in 200 μl phosphate-buffered saline (PBS) and complete Freund's adjuvant (1:1) intraperitoneally. Two weeks later the injection was repeated but incomplete Freund's adjuvant was used. On day 24 the tail blood from immunized mice was collected and tested by comparison with similar dilutions of normal mouse serum in an ELISA. Best responders were boosted by 20 μg antigen in 100 μl PBS intravenously and the same amount subcutaneously. On the third day mice were sacrified and in the case of the traditional technique splenocytes fusion was performed by the method of Davidson and Gerland (1977). The ratio of spleen cells to myeloma cells (Sp2/0) was 10:1. For our novel method the B-cell population were enriched and for four days in vitro growth stimulated as described above. The fusion was performed with a spleenocytes to myeloma cell ratio of 1:1 using polyethylene glycol at a concentration of 50%. After hybridization the cells were plated in 96-well plates and maintained in RPMI 1640 containing 10% fetal calf serum (FCS), 100 IU penicillin, 100 μg/ml streptomycin and HAT. The screening of hybridomas was performed by ELISA.

ELISA:

Microtiter plates (Nunc, Wiesbaden, Germany) were coated overnight at 4° C. with 100 μl antigen (10 μg/ml PBS). After washing and blocking with 350 μl PBS containing 3% bovine serum albumin (BSA), 150 μl of the hybridoma supernatants were incubated for 4 h at 4° C. The specific bound mAbs were labeled by rabbit anti-mouse IgG antibody (Jackson ImmunoResearch Lab.) coupled to peroxidase (HRP). o-phenylene diamine (Sigma) served as a substrate in the peroxidase assay. The reaction was stopped with 20 μl of H₂SO₄ and the optical density (OD) was measured with an ELISA-reader (THERMOmax, Molecular Devices) at 450 nm.

Results

The results will be described in connection with the accompanying drawings, in which

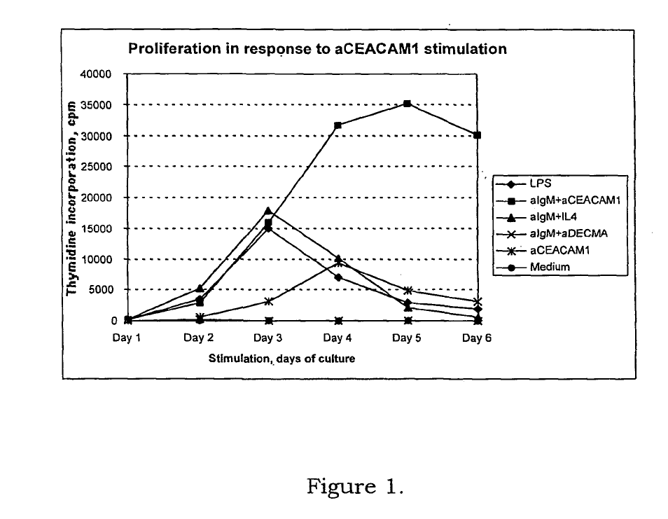

FIG. 1 is a graph showing proliferation of B lymphocytes in response to different stimulations; and

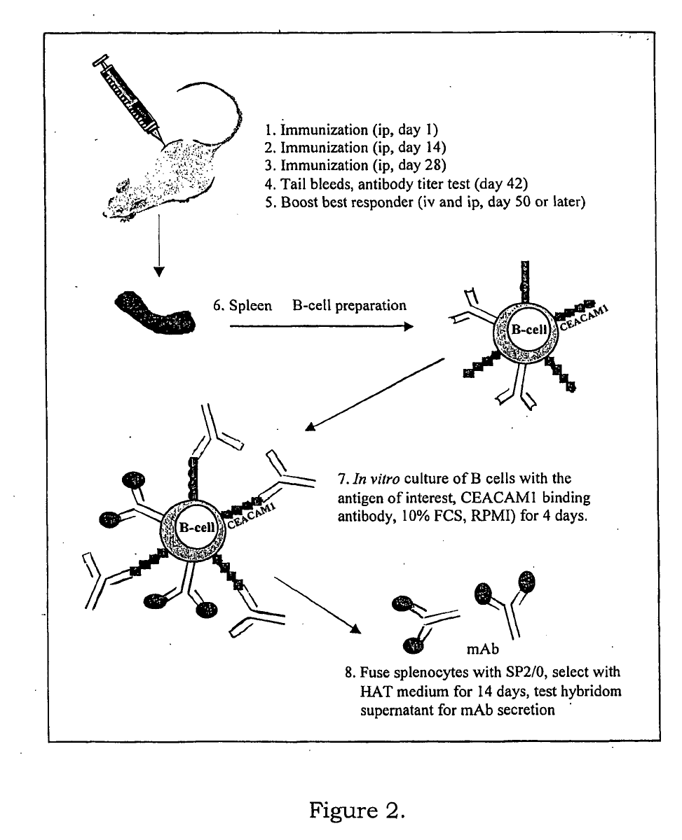

FIG. 2 is a schematic view of the different steps for production of monoclonal antibodies using aCAECAM1.

CEACAM1 is a BCR Dependent co-Regulator of B Cell Proliferation

Analysis of CEACAM1 has demonstrated that it is involved in BCR dependent stimulation of B lymphocytes (FIG. 1). The addition of anti-CEACAM1 antibody AgB10 and anti-IgM antibody to B cells isolated from spleen with a purity of >95% led to a strong induction of B cell proliferation. Furthermore, this stimulatory effect was drastically prolonged compared to LPS and anti-IgM plus interleulkin-4 (IL-4). In mice LPS, a thymus-independent mitogen, as well as anti-IgM plus interleukin-4 have been previously shown to induce polyclonal B cell proliferation. While the antigen dependent co-stimulation of CEACAM1 reaches its maximal effect after 4-5 days, all three control groups showed the highest proliferative induction after 3 days. On day 6 no proliferation could be detected in the groups of the known B cell stimulators, whereas anti-CEACAM1 together with anti-IgM still induced cell cycle progression. Neither anti-CEACAM1 mAb alone, nor the isotype control Decma1 together with anti-IgM had any pronounced effect on B cell proliferation. In principle, the binding of anti-IgM is mimicking the recognition of an appropriate antigen by the BCR. We analyzed the co-stimulatory effect mediated by CEACAM1 in B cells isolated from an RNAse A-immunized mouse. The results showed that CEACAM1 plays an important role in the antigen dependent activation of B cell proliferation.

Use of the Co-Stimulatory Effect of CEACAM1 for the Generation of mAbs

Utilizing a combination of in vivo and in vitro immunization a method was developed, providing mAbs with a higher efficiency compared to traditional techniques. The impact the method according to the invention was analyzed by generating maAbs specific for three different antigens: RNAse A, DNAse I and papain. Therefore, the Balb/c mice were handled according to the immunization scheme shown in FIG. 2. The spleenocytes of mice with a high serum titer of specific antibodies were isolated and separated into two parts. One part of the cells was fused with Sp2/0, further referred to as traditional method. B cells from another part of the spleenocytes were furthermore purified and cultured in vitro either with the antigen independent B-cell activator LPS or with anti-CEACAM1 mAb plus the appropriate antigen. Supernatants of hybridoma clones were screened for specific mAb secretion using a solid-phase ELISA. The results are summarized in table 1, 2a, 2b and 3 below.

TABLE 1
Fusion on day 4

| | | Number of | |
|---|---|---|---|
| | Total number | specific mAb | |
| | of grown | positive | Efficiency [%] = |
| | hybridoma [1] | hybridoma [2] | [2]/[1] × 100 |
| Traditional | 129 | 6 | 4.6% |
| method | | | |
| LPS | 8 | 1 | 12.5% |
| Anti-CEACAM1 + | 5 | 3 | 40% |
| RNAse A | | | |

TABLE 2a
Fusion on day 4

| | | Number of | |
|---|---|---|---|
| | Total number | specific mAb | |
| | of grown | positive | Efficiency [%] = |
| | hybridoma [1] | hybridoma [2] | [2]/[1] × 10 |
| Traditional | 82 | 3 | 3.7% |
| method | | | |
| LPS | 79 | 14 | 17.7% |
| Anti-CEACAM1 + | 68 | 27 | 39.7% |
| DNAse I | | | |

TABLE 2b
Fusion on day 6

| | | Number of | |
|---|---|---|---|
| | Total number | specific mAb | |
| | of grown | positive | Efficiency [%] = |
| | hybridoma [1] | hybridoma [2] | [2]/[1] × 10 |
| LPS | 27 | 3 | 11.1% |
| Anti-CEACAM1 + | 37 | 6 | 16.2% |
| DNAse I | | | |

TABLE 3
Fusion on day 4

| | | Number of | |
|---|---|---|---|
| | Total number | specific mAb | |
| | of grown | positive | Efficiency [%] = |
| | hybridoma [1] | hybridoma [2] | [2]/[1] × 10 |
| LPS | 37 | 7 | 18.9 |
| Anti-CEACAM1 + | 64 | 18 | 28.1 |
| papain | | | |

Using RNAse A, DNAse I and papain as antigens, it was possible to generate specific antibody secreting hybridoma cells (Table 1, 2a, 2b and 3). The efficiency was drastically increased if in vitro stimulation followed the in vivo immunization. Hereby, the antigen independent stimulus LPS already significantly increased the effectiveness of the in vivo/in vitro technique compared to the traditional method (Table 1 and 2). However, the antigen dependent co-activation triggered by CEACAM1 revealed an additional improvement of the efficiency compared to both the traditional method and the LPS in-vitro activation approach (Table 1, 2a, 2b and 3). Hybridization of B-lymphocytes with myeloma cells after 4 days of in vitro culture was clearly more sufficient than the fusion after 6 days with respect to both, the number of hybridoma clones and their mAb production efficiency. The fusion of B cell populations cultivated in either anti-CEACAM1 mAb alone or antigen alone did not give rise to a relevant number of hybridoma (data not shown).

Thus, the present invention provides an efficient method for the generation of mAbs by employing activating receptor molecules expressed on B-lymphocytes. The B cell proliferation assays (FIG. 1) showed a maximal effect for the LPS as well as for anti-IgM plus IL-4 treatment after 3 days of culture. After that the induced proliferation rapidly decreased. No significant B cell activation could be detected on day 6. The anti-CEACAM1 plus anti-IgM stimulation increased with the same amplitude like the control groups until day 3 but thereafter increased tremendously up to day 4-5. Surprisingly, CEACAM1 triggered B cell proliferation continued to a very high extent. However, because of technical reasons a later time point than 6 days was not measured.

Using B cells, which were isolated from immunized mice we could demonstrate that the substitution of anti-IgM antibody with a specific antigen during the in vitro cultivation revealed the tendency of an antigen dependent co-stimulation triggered by CEACAM1. Thus, the immune response can be induced by CEACAM1 after BCR engagement and leads to a clonal expansion of antigen specific B cell populations. This effect was employed in the method for mAb production according to the invention. Although the anti-CEACAM1/antigen approach was more productive compared to the LPS stimulation procedure, both methods are valuable improvements for the future production of mAbs.

In contrast to the LPS treatment, CEACAM1 induces an antigen dependent B cell stimulation and can therefore be used for an only in vitro immunization approach, which is a primary, antigen-specific B-cell activation. Hereby, the antigen-specific activation of mouse cells is supported by a cocktail of lymphokines derived from normal T helper cells and from a murine T thymoma cell line (Harlow and Lane 1988). The advantages compared to the conventional in vivo immunization method is that it takes only five days compared to normally several months when in vivo methods are utilized. mAbs have been produced against phylogenetically very conserved structures like calmodulin, actin, and histones as well as against allogeneic and syngeneic proteins. This makes the in vitro immunizations to a potentially powerful technology. However, most of the produced antibodies are from the IgM class in known in vitro methods. In contrast, the BCR co-activator CEACAM1 induced not only a prolonged antigen specific B cell proliferation but also facilitates class switching to IgG1, IgG2b and IgG3 by activating germline promotors. However, it is not sufficient by itself but combined with LPS or other agents. Therefore, the present invention also contemplates a novel in vitro immunization approach based on the CEACAM1 effect on B cells.

### REFERENCES

Davidson R. L. and Gerald P. S.: Induction of mammalian somatic cell hybridization by polyethylene glycol. Methods Cell Biol. 15: 325-338, 1977.

Green L. L.: Antibody engineering via genetic engineering of the mouse: XenoMouse strains are a vehicel for the faclie generation of therapeutic human monoclonal antibodies. J Immunol Methods 23111-23, 1999.

Harlow E. and Lane D.: Antibodies, a laboratory manual. Cold Spring Harbor Laboratory 1988.

Kammerer R., Hahn S., Singer B. B., Luo J. S. and von Kieist S.: Biliary glycoprotein (CD66a), a cell adhesion molecule of the immunoglobulin superfamily, on human lymphocytes: structure, expression and involvement in T cell activation. Eur. J. Immunol. 28: 3664-3674, 1998.

Karasuyama H. and Melchers F.: Establishment of mouse cell lines which constitutively secrete large quantities of interleukin 2, 3, 4 or 5, using modified cDNA expression vectors. Eur. J. Immunol. 18:97, 1988.

Köhler G. and Milstein S.: Continuous culture of fused cells secreting antibody of redefined specificity. Nature 256, 495, 1975.

Kuprina N. N., Baranov V. N., Yazova A. K., Rudinskaya T. D., Escribano M., Cordier J., Gleiberman A. S., and Goussev A. I.: The antigen of bile canaliculi of the mouse hepatocyte: identification and ultrastructural localization. Histochemistry 94: 179-186, 1990.

Leptin M., Potash M. J., Grützmann R., Heusser C., Shulman M., Köhler G. and Melchers F.: Monoclonal antibodies specific for murine IgM. I. Characterization of antigenic determinants on the four constant domains of the m heavy chain. Eur. J. Immunol. 14:534, 1984.

Singer B. B., Scheffrahn I. and Öbrink B.: The tumor growth-inhibiting cell adhesion molecule CEACAM1 (C-CAM) is differently expressed in proliferating and quiescent epithelial cells and regulates cell proliferation. Cancer Research 60: 1236-1244, 2000.

Beauchemin et al: Redefined nomenclature for members of the carcinoembryonic antigen family. Exp. Cell Res. 252: 243-249, 1999.

1. A method for production of monoclonal antibodies, comprising immunization of an animal with an antigen for enrichment of antibody producing B cells; preparation of B cells; fusion of B cells with immortal cells to form antibody producing hybridomas, comprising the following additional step:
in vitro expansion of said B cells in the presence of B cell stimulating agent(s) before said fusion, wherein said B cell stimulating agent(s) comprises an antibody against CEACAM, or CEACAM ligands or against a B cell stimulatory variant thereof, and an antigen against which said B cells are reactive.

2. A method according to claim 1, wherein said B cell stimulating agent is an antibody against CEACAM1.

3. A method according to claims 1 or 2, wherein said immunization is in vivo.

4. A method according to claims 1 or 2, wherein said immunization is in vitro.

5. A kit for production of monoclonal antibodies, comprising B cell stimulating agent(s) for in vitro expansion of B cells.

6. A kit according to claim 5, wherein said B cell stimulating agent(s) comprises anti-CEACAM antibody.

7. A kit according to claim 6, wherein said B cell stimulating agent(s) comprises anti-CEACAM1 antibody.

8. Use of CEACAM in the production of a drug for B cell stimulation.

9. Use according to claim 8, of CAECAM1 antibodies and ligands as pharmaceutical compositions or vaccines for immuno-treatment of a subject.

10. A method for augmenting an immune response in a patient comprising the step of administering an amount of CEACAM, anti-CEACAM antibody and CEACAM ligand to the patient sufficient to generate an increase in the number of the patient's B cells.

11. A method according to claim 10, further comprising the step of administering one or more of the molecules selected from the group consisting of GM-CSF, IL-4, TNFa, IL-3, c-kit ligand, flt-ligand and fusions of GM-CSF and IL-3.

12. A method of enhancing a mammal's immune response to a vaccine antigen, comprising the steps of administering to such mammal an immunogenic amount of the vaccine antigen and an immunogenicity-augmenting amount of CEACAM, anti-CEACAM antibody and CEACAM ligand in concurrent or sequential combination with such vaccine antigen.

13. A vaccine adjuvant comprising a molecule selected from the group consisting of CEACAM, anti-CEACAM antibody and CEACAM ligand.
